(19) United States
(12) Patent Application Publication
(10) Pub. No.: US 2003/0074013 A1
(43) Pub. Date: Apr. 17, 2003

(54) Scalpel protective guard

(76) Inventors: Paul Schooler, Fraser, MI (US); Michael Rottman, West Bloomfield, MI (US)

Correspondence Address: CARLSON, GASKEY & OLDS, P.C., 400 WEST MAPLE ROAD, SUITE 350, BIRMINGHAM, MI, 48009, US

(21) Appl. No.: 09/981,032

(22) Filed: Oct. 17, 2001

(51) Int. Cl. A61B 17/32

(52) U.S. Cl. 606/167

(57) ABSTRACT

A surgical blade assembly comprises a scalpel blade and handle. A guard is attached to the assembly and is pivotally moveable between a sheathed and an unsheathed position. A spring resiliently biases the guard to the sheathed position. A hinge may attach the guard to the assembly and allow pivoting of the guard relative to the blade.

### BACKGROUND OF THE INVENTION

The invention relates to a protective guard for a scalpel.

A surgical scalpel typically comprises a blade and a handle. The blade is extremely sharp to permit a surgeon to cut into the skin of a patient. The sharpness of a blade, however, poses a potential hazard to individuals handling the instrument. Not only may the blade accidentally cut any handler but may expose him to pathogens, germs, and viruses from the blood of the patient.

Recently, scalpels have been provided with removable covers. The covers are removed prior to use in surgery, and are recommended to be maintained near the location of the surgery. Once the scalpel is done being utilized as part of the surgery, the cover may be replaced on the scalpel.

There are certain disadvantages with the above approach. First, the cover must be found and again placed back on the scalpel. This may not always occur. Moreover, in the rush of surgery it is sometimes the case that the scalpel is not recovered for a period of time. During this period of time the scalpel provides a risk of cutting.

In addition, the placement of the cover back onto the scalpel is itself dangerous. It is possible to cut oneself while putting the cover onto the scalpel and thus the prior art removable scalpel is somewhat disadvantageous.

A need therefore exists for a protective cover for a scalpel blade that permits a surgeon to use a scalpel without interference and still provides protection during the course of an operation.

### SUMMARY OF THE INVENTION

Like existing scalpels, the invention comprises a scalpel blade with a handle. In contrast, however, the invention employs a guard that is attached to the scalpel and is moveable between a sheathed and an unsheathed position. The guard maybe resiliently biased toward the sheathed position to protect against cuts during surgery as well as after. The guard always remains with the scalpel but does not interfere with its use.

The guard is hinged to the handle and pivots on this hinge between the sheathed position and the unsheathed position. A spring serves to urge the guard towards the sheathed position. The spring is received between the guard and the handle in a base formed in the handle.

In a preferred embodiment, the spring is positioned at an angle relative to the top of the body of the scalpel handle. This angle ensures that the hood is biased about the pivot pin to the sheaved position.

The hood is preferably provided with a thumb indentation that the surgeon can contact with any portion of the hand to move the hood to its open position. Thus, the surgeon merely biases the hood against the force of the spring, and the hood is thus moved back to its unsheathed position for surgery.

Once the surgeon releases the hood the hood will immediately spring back to the sheathed position under the influence of the spring. Thus, there is no need to replace the cover, as it will immediately and without any action from the surgeon return to the sheathed position.

In a preferred embodiment of this invention, the hood has a shape such that a thicker portion of the hood is found at the handle, and perhaps a portion of the scalpel blade more remote from the cutting end of the scalpel. The hood preferably becomes thin at forward positions. This will facilitate the surgeon being able to see more clearly around the hood at the location of the surgery.

In addition, to provide the same benefit, the guard may be clear. The guard may be sized to receive the blade such that the guard covers the entire surface of the blade, preventing the exposure of any sharp edge except when the scalpel is used.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1A illustrates an embodiment of the invention, showing scalpel blade, body, and guard.

FIG. 1B shows a detail of the FIG. 1A embodiment.

FIG. 2 illustrates the bottom side of the guard of FIG. 1.

FIG. 3 illustrates the top side of the guard, highlighting its tapered form.

FIG. 4 illustrates the embodiment of the invention of FIG. 1, illustrating the pivoting of the guard relative to the scalpel blade.

### DETAILED DESCRIPTION OF THE PREFERRED EMBODIMENT

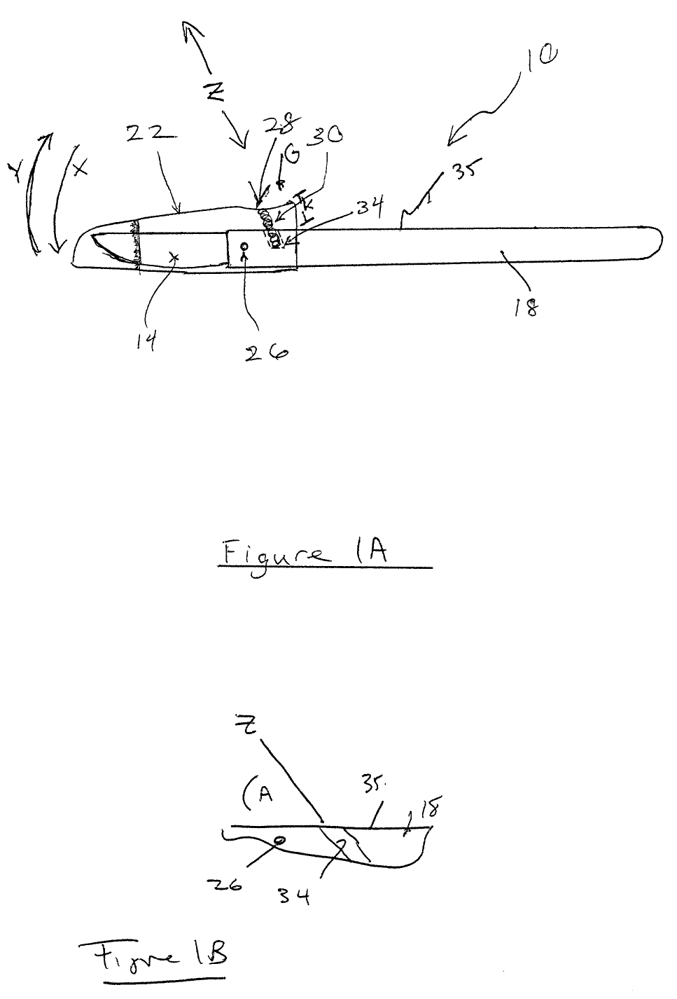

FIG. 1A illustrates an embodiment of the invention, surgical blade assembly 10, comprising scalpel blade 14, body 18, and guard 22. As shown, guard 22 is mounted to handle body 18, through a hinge pin 26. Scalpel blade 14 is attached to body 18 as known. Additionally, spring 30, shown in relatively uncompressed state, in hole 34 of body 18 urges guard 22 to be in a closed state, or sheathed, as shown in FIG. 1. One end of spring 30 is fixed within guard 22. Spring is also inclined along an Z axis in hole 34 relative to a top plane 35 of the body 18.

Space K between guard 22 and body 18 creates room for guard 22 to pivot relative to scalpel blade 14 in the direction of arrow Y to an open state or unsheathed position. In this state, spring 30 is compressed. A force in the direction of arrow G will move guard in this direction compressing spring 30 and making space K smaller as guard 22 pivots on hinge 26. Preferably, the area 28 on top of the hood 22 receives a finger or other portion of the surgeon's hand. The force is passed through this area down against the spring to bias the spring to its unsheathed position. Once the surgeon releases the hood, the spring will return the hood to its sheathed position. One of ordinary skill in the art would know of other techniques to resiliently bias guard 22.

As shown in FIG. 1B, the pivot pin 26 is spaced to one side of the hole 34. The hole 34 is spaced at an angle A along the axis Z relative to the top surface 35 of the handle body 18. In this way, the force from the spring will cause the hood to pivot counter-clockwise, as shown in this view, about the hinge pin 26. This will provide a sure and prompt closing of the hood once released.

FIG. 2 illustrates a bottom view of guard 22. Guard 22 has slot 42 to at least partially receive scalpel blade 14. Slot 42 is accordingly sized to accommodate scalpel blade 14. Edge 21 of guard 22 is blunt.

FIG. 2 also shows tapered portion 38. Accordingly, guard 22 comprises first end 40 having a width W1 and second end 41 having a width W2. To taper guard 22, width W1 is less than the width of W2. This taper offers an improved view of the tip of surgical blade assembly 10 so that a surgeon may track and follow scalpel blade 14 in a less obstructed fashion.

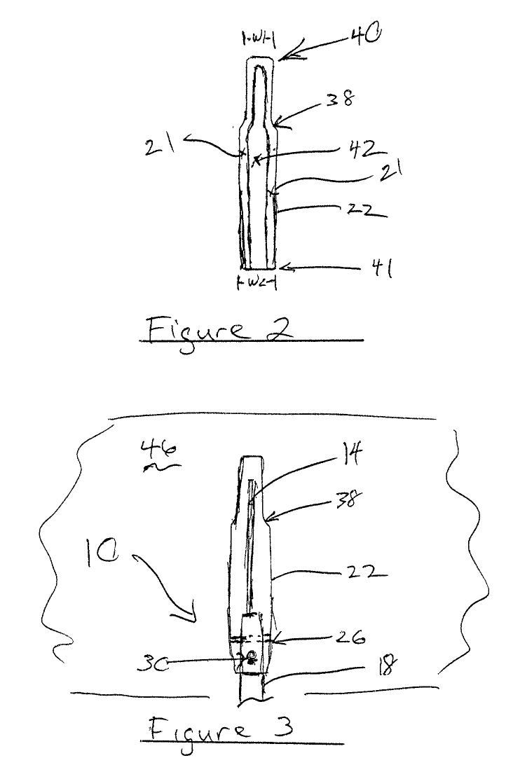

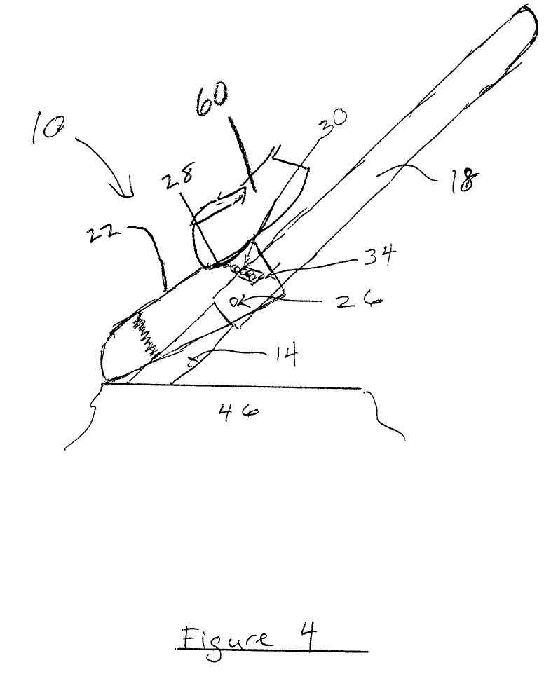

Indeed, FIG. 3 illustrates a view of surgical blade assembly 10 as possibly seen by a surgeon using the instrument. As shown, tapered portion 38 offers less sight obstruction to the surgeon of cutting surface 46. Also, guard 22 may be made of generally transparent plastic to permit a direct view of scalpel blade 14 on cutting surface 46 as shown.

FIG. 4 illustrates surgical instrument 10 on cutting surface 46. As shown, scalpel blade 14 cuts into surface 46. Guard 22 pivots relative to scalpel blade 14 to permit it to cut into cutting surface 46 without interference. As mentioned above, a surgeon may place a finger 60 on the area 28 and cause the hood to pivot relative to the handle 18. This allows the scalpel blade 14 to be exposed for cutting. Once the surgeon releases the hood, the spring 30 will return the hood to its sheathed position such as shown in FIG. 1A.

In this way, surgical blade assembly has a guard that is easily moved out of the way of scalpel blade 14 as it is used. When guard 22 is released, it is then closed by spring 30 as it decompresses. Thus, without any additional thought or effort, guard 22 protects against accidental cuts.

The aforementioned description is exemplary rather that limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. A surgical blade assembly comprising:
a scalpel blade;
a body operatively connected to said scalpel blade; and
a guard attached to said body and movable between an open position and a closed position wherein said closed position at least partially covers said scalpel blade.

2. A surgical blade as set forth in claim 1 wherein said guard is resiliently biased towards said closed position.

3. The surgical blade assembly of claim 2 wherein said guard pivots relative to said scalpel blade between said open position and said closed position.

4. The surgical blade assembly of claim 3 wherein a spring resiliently biases said guard towards said closed position.

5. The surgical blade assembly of claim 4 wherein said spring is positioned in said handle to extend at an angle relative to a top surface of said handle and such that said hood is caused to pivot about a pivot point.

6. The surgical blade assembly of claim 5 wherein said spring is spaced relative to said hinge point to be on an opposed side of said hinge point relative to said blade.

7. The surgical blade assembly of claim 1 wherein said guard has a thinner portion and a thicker portion, with said thicker portion being spaced from a forward end of said scalpel blade, and said thinner portion being positioned adjacent said forward end of said scalpel blade to minimize the obstruction of a surgeon's view.

8. The surgical blade assembly of claim 1 wherein said guard is formed of a generally transparent plastic.

9. A surgical blade assembly comprising:
a scalpel blade;
a body operatively connected to said scalpel blade;
a guard attached to said body and pivotally movable between an open position and a closed position wherein said closed position at least partially covers said scalpel blade; and
a spring resiliently biasing said guard towards said closed position.

10. A surgical blade assembly as set forth in claim 9 wherein said guard is resiliently biased towards said closed position.

11. The surgical blade assembly of claim 10 wherein said guard pivots relative to said scalpel blade between said open position and said closed position.

12. The surgical blade assembly of claim 9 wherein said guard has a thinner portion and a thicker portion, with said thicker portion being spaced from a forward end of said scalpel blade, and said thinner portion being positioned adjacent said forward end of said scalpel blade to minimize the obstruction of a surgeon's view.

13. The surgical blade assembly of claim 9 wherein said guard is formed of a generally transparent plastic.
